(19) United States
(12) Patent Application Publication
(10) Pub. No.: US 2022/0186200 A1
(43) Pub. Date: Jun. 16, 2022

(54) AMYLASES AND METHODS FOR MAKING AND USING THEM

(71) Applicant: BASF SE, Ludwigshafen am Rhein (DE)

(72) Inventors: Hugo Urbina, San Diego, CA (US); Kristine W. Chang, San Diego, CA (US); Tong Li, San Diego, CA (US); Xuqiu Tan, San Diego, CA (US); Jared Dennis, San Diego, CA (US); Jochen Kutscher, San Diego, CA (US); Adrienne Huston Davenport, San Diego, CA (US)

(21) Appl. No.: 17/438,088

(22) PCT Filed: Mar. 10, 2020
(86) PCT No.: PCT/US2020/021818

§ 371 Date: Sep. 10, 2021

Related U.S. Application Data
(60) Provisional application No. 62/816,661, filed on Mar. 11, 2019.

(51) Int. Cl. C12N 9/26

(52) U.S. Cl. CPC C12N 9/2411

(57) ABSTRACT

Variant polypeptides having G4-amylase activity and methods of making and using the enzymes in baking, detergents, personal care products, in the processing of textiles, in pulp and paper processing, in the production of ethanol, lignocellulosic ethanol, or syrups; or as viscosity breakers in oilfield and mining industries.

### CROSS-REFERENCE TO RELATED APPLICATIONS

This application claims the benefit of and priority to U.S. Provisional Application No. 62/816,661, filed on Mar. 11, 2019, the contents of which are incorporated herein by reference in their entirety.

### SEQUENCE LISTING

This application includes a nucleotide and amino acid sequence listing in computer readable form (CRF) as an ASC II text (.txt) file according to “Standard for the Presentation of Nucleotide and Amino Acid Sequence Listings in International Patent Applications Under the Patent Cooperation Treaty (PCT)” ST.25. The sequence listing is identified below and is hereby incorporated by reference into the specification of this application in its entirety and for all purposes.

| | Date of | Size |
|---|---|---|
| File Name | Creation | (bytes) |
| 171247_SequenceListingForFiling.txt | Mar. 7, 2019 | 6.33 KB |

### BACKGROUND

### Field

The present disclosure relates generally to molecular and cellular biology and biochemistry. More specifically, the disclosure relates to polypeptides having amylase activity, polynucleotides comprising the coding sequences for these polypeptides, and methods for making and using these polypeptides and polynucleotides.

### Description of the Related Art

Amylase is an enzyme that catalyzes the hydrolysis of starches into sugars. Amylases hydrolyze internal α-1,4glucosidic linkages in starch, largely at random, to produce smaller molecular weight malto-dextrins. The breakdown of starch is important in the digestive system and commercially. Amylases are of considerable commercial value, being used in the initial stages (liquefaction) of starch processing; in wet corn milling; in alcohol production; as cleaning agents in detergent matrices; in the textile industry for starch desizing; in baking applications; in the beverage industry; in oilfields in drilling processes; in inking of recycled paper; and in animal feed.

Amylases are produced by a wide variety of microorganisms including Bacillus and Aspergillus, with most commercial amylases being produced from bacterial sources such as Bacillus licheniformis, Bacillus amyloliquefaciens, Bacillus subtilis, or Bacillus stearothermophilus. In recent years, the enzymes in commercial use have been those from Bacillus licheniformis because of their heat stability and performance, at least at neutral and mildly alkaline pHs.

There is a need for an improved amylase that helps in improving bread volume and for anti-staling, as well as a better enzyme for other uses like animal feed, detergents, personal care products, processing of textiles, pulp and paper processing, ethanol production, beverage industry, and as viscosity breakers in oilfield and mining industries. More particularly there is a need for an improved G4-amylase for use as an industrial enzyme.

### BRIEF SUMMARY OF THE INVENTION

In one embodiment, a variant polypeptide having G4 anylase activity selected from the group consisting of: a polypeptide having at least 80% sequence identity with SEQ ID NO: 1; a polypeptide encoded by a polynucleotide having at least 80% sequence identity with SEQ ID NO: 2; a polypeptide encoded by a polynucleotide that hybridizes under high stringency conditions with (i) a polynucleotide that encodes the amino acid sequence of SEQ ID NO: 1, or (ii) the full-length complement of (i); a variant of the polypeptide of SEQ ID NO: 1 comprising a modification at one or more, and not exceeding 36 positions and having G4 amylase activity; a polypeptide encoded by a polynucleotide that differs from SEQ ID NO: 1 due to the degeneracy of the genetic code; and a fragment of the polypeptide (a), (b), (c), (d) or (e) having G4 amylase activity; wherein the variant amylase has G4 amylase activity, is provided.

In one embodiment, the modification is a substitution, deletion, and/or insertion of amino acids. In one embodiment, the modification is at an amino acid residue position number: 31, 111, 123, 136, 141, 143, 145, 148, 149, 152, 153, 156, 160, 162, 178, 181, 184, 190, 206, 213, 215, 227, 309, 315, 345, 346, 348, 394, 401, 422, or any combination thereof. In one embodiment, the modification is a substitution. In one embodiment, the modification is an amino acid substitution selected from T31C, P111T, G123F, G136M, G136R, G136K, G136C, D141M, D141I, D141P, T143E, T143P, T143A, T143Y, T143F, P145Y, Y148W, A149P, C152P, C152T, D153S, D156G, G160N, G160M, D162E, D178N, A181E, A181N, A181R, R184A, G190R, A206D, H213D, N215P, G227S, G227D, G227H, G227E, G227N, G227W, G227Q, H309R, H309K, A315S, A315C, A315Y, D345E, F346L, R348G, S394E, S401Y, Q422R, or any combinations thereof.

In one embodiment, a variant polypeptide with an amino acid sequence that is at least 80% identical, at least 80.5%, at least 81%, at least 81.5%, at least 82%, at least 82.5%, at least 83%, at least 83.5%, at least 84%, at least 84.5%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or at least 100% identical to the amino acid sequence as set forth in SEQ ID NO:1, is provided.

In one embodiment, a variant polypeptide encoded by a polynucleotide having at least 80% identical, at least 80.5%, at least 81%, at least 81.5%, at least 82%, at least 82.5%, at least 83%, at least 83.5%, at least 84%, at least 84.5%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or at least 100% identical to the SEQ ID NO: 2, is provided.

In one embodiment, the variant polypeptide has an increase in enzyme activity, thermostability, or any combination thereof when compared to the G4-amylase of SEQ ID NO:1. In one embodiment, the variant polypeptide having at least 5% higher residual activity at at 65° C. in comparison with Seq 1 is provided.

In one embodiment, a variant polypeptide having G-4-amylase activity is provided, wherein the variant polypeptide is an amino acid sequence that is at least 80% identical, at least 80.5%, at least 81%, at least 81.5%, at least 82%, at least 82.5%, at least 83%, at least 83.5%, at least 84%, at least 84.5%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or at least 100% identical to the amino acid sequence as set forth in SEQ ID NO:1, and the variant polypeptide has an increase in enzyme activity, thermostability, pH-stability, or any combination thereof when compared to the G4-amylase of SEQ ID NO:1.

In one embodiment, the variant polypeptide is a hybrid of at least one variant polypeptide having G4 amylase activity, and a second polypeptide having amylase activity, wherein the hybrid has G4-amylase activity.

In one embodiment, a composition comprising a variant polypeptide having G4 amylase activity is provided. In another embodiment, the composition comprises as a variant polypeptide having G4 amylase activity and a second enzyme. In one embodiment, the second enzyme is selected from the group consisting of: a second G4-amylase, a lipase, an alpha-amylase, a beta-amylase, a xylanase, a protease, a cellulase, a glucoamylase, an oxidoreductase, a phospholipase, and a cyclodextrin glucanotransferase.

In one embodiment, a method of making a variant polypeptide is provided. The method comprises: providing a template nucleic acid sequence encoding the variant polypeptide, transforming the template nucleic acid sequence into an expression host, cultivating the expression host to produce the variant polypeptide, and purifying the variant polypeptide. In the method, the template nucleic acid is a variant nucleotide of the nucleic acid sequence as set forth in SEQ ID NO:2, wherein the variant nucleotide is a nucleic acid sequence that is at least 80% identical to the nucleic acid sequence as set forth in SEQ ID NO:2, and wherein the variant nucleotide encodes a polypeptide having G4-amylase activity. In one embodiment, the expression host is selected from the group consisting of: a bacterial expression system, a yeast expression system, a fungal expression system, and a synthetic expression system. The bacterial expression system is selected from an E. coli, a Bacillus, a Pseudomonas, and a Streptomyces. The yeast expression system is selected from a Candida, a Komagataella, a Saccharomyces, a Schizosaccharomyces. The fungal expression system is selected from a Penicillium, an Aspergillus, a Fusarium, a Myceliopthora, a Rhizomucor, a Rhizopus, a Thermomyces, and a Trichoderma.

In one embodiment, a method of preparing a dough or a baked product prepared from the dough, comprising adding one of the variant polypeptides to the dough and baking it is provided.

In one embodiment, a method of use of the variant polypeptide for processing starch is provided.

In one embodiment, a method of use of the variant polypeptide for cleaning or washing textiles, hard surfaces, or dishes, is provided. In one embodiment, a method of use of the variant polypeptide for making ethanol is provided. In one embodiment, a method of use of the variant polypeptide for treating an oil well is provided. In one embodiment, a method of use of the variant polypeptide for processing pulp or paper is provided. In one embodiment, a method of use of the variant polypeptide for feeding an animal is provided. In one embodiment, a method of use of the variant polypeptide for making syrup is provided.

Other objects, advantages and features of the present disclosure will become apparent from the following specification.

### DETAILED DESCRIPTION OF THE INVENTION

All patents, applications, published applications and other publications referred to herein are incorporated by reference for the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

As used herein, the singular forms “a”, “an”, and “the” include plural references unless indicated otherwise, expressly or by context. For example, “a” dimer includes one or more dimers, unless indicated otherwise, expressly or by context.

The term “amplification” (“a polymerase extension reaction”) means that the number of copies of a polynucleotide is increased.

As used herein, “sequence identity” or “identity” in the context of two protein sequences (or nucleotide sequences) includes reference to the residues in the two sequences which are the same when aligned for maximum correspondence over a specified comparison window.

Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this description is that alignment, from which the highest sequence identity can be determined.

After aligning two sequences, in a second step, an identity value is determined from the alignment produced. For purposes of this description, percent identity is calculated by:

%-identity=(identical residues/length of the alignment region which is showing the respective sequence of this description over its complete length)*100.

Thus, sequence identity in relation to comparison of two amino acid sequences according to this embodiment is calculated by dividing the number of identical residues by the length of the alignment region which is showing the respective sequence of this description over its complete length. This value is multiplied with 100 to give “%-identity”.

For calculating the percent identity of two DNA sequences the same applies as for the calculation of percent identity of two amino acid sequences with some specifications. For DNA sequences encoding for a protein the pairwise alignment shall be made over the complete length of the coding region from start to stop codon excluding introns. Introns, present in the other sequence, so the sequence to which the sequence of this description is compared, may also be removed for the pairwise alignment. Percent identity is then calculated by: %-identity=(identical residues/length of the alignment region which is showing the coding region of the sequence of this description from start to stop codon excluding introns over its complete length)*100.

For non-protein-coding DNA sequences the pairwise alignment shall be made over the complete length of the sequence of this description, so percent identity is calculated by:

%-identity=(identical residues/length of the alignment region which is showing the sequence of this description over its complete length)*100.

Moreover, the preferred alignment program implementing the Needleman and Wunsch algorithm (J. Mol. Biol. (1979) 48, p. 443-453) is “NEEDLE” (The European Molecular Biology Open Software Suite (EMBOSS)) with the programs default parameters (gapopen=10.0, gapextend=0.5 and matrix=EDNAFULL).

Sequences, having identical or similar regions with a sequence of this description, and which shall be compared with a sequence of this description to determine % identity, can easily be identified by various ways that are within the skill in the art, for instance, using publicly available computer methods and programs such as BLAST, BLAST-2, available for example at NCBI.

Variants of the parent enzyme molecules may have an amino acid sequence which is at least n percent identical to the amino acid sequence of the respective parent enzyme having enzymatic activity with n being a number or an integer between 50 and 100, preferably 50, 55, 60, 65, 70, 75, 80, 85, 90, 91, 92, 93, 94, 95, 96, 97, 98 or 99 compared to the full length polypeptide sequence. Preferably, variant enzymes which are n percent identical when compared to a parent enzyme, have enzymatic activity.

Enzyme variants may be defined by their sequence similarity when compared to a parent enzyme. Sequence similarity usually is provided as “% sequence similarity” or “%-similarity”. For calculating sequence similarity in a first step a sequence alignment has to be generated as described above. In a second step, the percent-similarity has to be calculated, whereas percent sequence similarity takes into account that defined sets of amino acids share similar properties, e.g., by their size, by their hydrophobicity, by their charge, or by other characteristics. Herein, the exchange of one amino acid with a similar amino acid is called “conservative mutation”. Enzyme variants comprising conservative mutations appear to have a minimal effect on protein folding resulting in certain enzyme properties being substantially maintained when compared to the enzyme properties of the parent enzyme.

For determination of %-similarity according to this description the following applies, which is also in accordance with the BLOSUM62 matrix as for example used by program “NEEDLE”, which is one of the most used amino acids similarity matrix for database searching and sequence alignments.
Amino acid A is similar to amino acids S;
Amino acid D is similar to amino acids E; N;
Amino acid E is similar to amino acids D; K; Q;
Amino acid F is similar to amino acids W; Y;
Amino acid H is similar to amino acids N; Y;
Amino acid I is similar to amino acids L; M; V;
Amino acid K is similar to amino acids E; Q; R;
Amino acid L is similar to amino acids I; M; V;
Amino acid M is similar to amino acids I; L; V;
Amino acid N is similar to amino acids D; H; S;
Amino acid Q is similar to amino acids E; K; R;
Amino acid R is similar to amino acids K; Q;
Amino acid S is similar to amino acids A; N; T;
Amino acid T is similar to amino acids S;
Amino acid V is similar to amino acids I; L; M;
Amino acid W is similar to amino acids F; Y; and
Amino acid Y is similar to amino acids F; H; W.

Conservative mutation (also referred to as conservative amino acid mutation) may occur over the full length of the sequence of a polypeptide sequence of a functional protein such as an enzyme. In one embodiment, such mutations are not pertaining the functional domains of an enzyme. In one embodiment, conservative mutations are not pertaining to the catalytic centers of an enzyme.

Therefore, according to the present description the following calculation of percent-similarity applies:

%-similarity=[(identical residues+similar residues)/length of the alignment region which is showing the respective sequence of this description over its complete length]*100.

Especially, variant enzymes comprising conservative mutations which are at least m % similar to the respective parent sequences with m being a number or an integer between 50 and 100, preferably 50, 55, 60, 65, 70, 75, 80, 85, 90, 91, 92, 93, 94, 95, 96, 97, 98 or 99 compared to the full-length polypeptide sequence, are expected to have essentially unchanged enzyme properties. Preferably, variant enzymes with m %-similarity when compared to a parent enzyme, have enzymatic activity.

Homologous refers to a gene, polypeptide, polynucleotide with a high degree of similarity, e.g. in position, structure, function or characteristic, but not necessarily with a high degree of sequence identity.

As used herein, “substantially complementary or substantially matched” means that two nucleic acid sequences have at least about 90% sequence identity. Preferably, the two nucleic acid sequences have at least, or at least about, 95%, 96%, 97%, 98%, 99%, or 100% of sequence identity. Alternatively, “substantially complementary or substantially matched” means that two nucleic acid sequences can hybridize under high stringency condition(s).

The term “hybridization” as defined herein is a process wherein substantially complementary nucleotide sequences anneal to each other. The hybridization process can occur entirely in solution, i.e. both complementary nucleic acids are in solution. The hybridization process can also occur with one of the complementary nucleic acids immobilized to a matrix such as magnetic beads, Sepharose beads or any other resin. The hybridization process can furthermore occur with one of the complementary nucleic acids immobilized to a solid support such as a nitro-cellulose or nylon membrane or immobilized by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids. Hybridization according to this description means, that hybridization must occur over complete length of the sequence of the invention. Such hybridization over the complete length, as defined herein, means, that when the sequence of this invention is fragmented into pieces of 300-500 bases, each fragment will hybridize.

The term “stringency” refers to the conditions under which a hybridization takes place. The stringency of hybridization is influenced by conditions such as temperature, salt concentration, ionic strength and hybridization buffer composition. Generally, low stringency conditions are selected to be about 30° C. lower than the thermal melting point (Tm) for the specific sequence at a defined ionic strength and ph. Medium stringency conditions are when the temperature is 20° C. below Tm, and high stringency conditions are when the temperature is 10° C. below Tm. High stringency hybridization conditions are typically used for isolating hybridizing sequences that have high sequence identity to the target nucleic acid sequence. However, nucleic acids may deviate in sequence and still encode a substantially identical polypeptide, due to the degeneracy of the genetic code. Therefore, medium stringency hybridization conditions may sometimes be needed to identify such nucleic acid molecules. The “Tm” is the temperature under defined ionic strength and pH, at which 50% of the target sequence hybridizes to a perfectly matched probe. The Tm is dependent upon the solution conditions and the base composition and length of the probe. For example, longer sequences hybridize specifically at higher temperatures. The maximum rate of hybridization is obtained from about 16° C. up to 32° C. below Tm. The presence of monovalent cations in the hybridization solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridization to be performed at 30 to 45° C., though the rate of hybridisation will be lowered. Base pair mismatches reduce the hybridization rate and the thermal stability of the duplexes. On average and for large probes, the Tm decreases about 1° C. per % base mismatch. The Tm may be calculated using the following equations, depending on the types of hybrids:
DNA-DNA hybrids (Meinkoth and Wahl, Anal. Biochem., 138: 267-284, 1984): Tₘ=81.5° C.+16.6×log[Na+]^{a}+0.41×%[G/C^{b}]−500×[L^{c}]⁻¹−0.61x % formamide
DNA-RNA or RNA-RNA hybrids: Tₘ=79.8+18.5 (log₁₀[Na+]^{a})+0.58 (% G/C^{b})+11.8 (% G/C^{b})²−820/L^{c}
oligo-DNA or oligo-RNA^{d} hybrids:
For <20 nucleotides: Tₘ=2 (lₙ)
For 20-35 nucleotides: Tₘ=22+1.46 (lₙ)
^{a} or for other monovalent cation, but only accurate in the 0.01-0.4 M range.
^{b} only accurate for % GC in the 30% to 75% range.
L=length of duplex in base pairs.
^{d} Oligo, oligonucleotide; ln, effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridization buffer, and treatment with RNase. For non-related probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridization and which will either maintain or change the stringency conditions.

Besides the hybridization conditions, specificity of hybridization typically also depends on the function of post-hybridization washes. To remove background resulting from non-specific hybridization, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridization stringency. A positive hybridization gives a signal that is at least twice of that of the background. Generally, suitable stringent conditions for nucleic acid hybridization assays or gene amplification detection procedures are as set forth above. More or less stringent conditions may also be selected. The skilled artisan is aware of various parameters which may be altered during washing and which will either maintain or change the stringency conditions.

For example, typical high stringency hybridization conditions for DNA hybrids longer than 50 nucleotides encompass hybridization at 65° C. in 1×SSC or at 42° C. in 1×SSC and 50% formamide, followed by washing at 65° C. in 0.3×SSC. Examples of medium stringency hybridization conditions for DNA hybrids longer than 50 nucleotides encompass hybridization at 50° C. in 4×SSC or at 40° C. in 6×SSC and 50% formamide, followed by washing at 50° C. in 2×SSC. The length of the hybrid is the anticipated length for the hybridizing nucleic acid. When nucleic acids of known sequence are hybridized, the hybrid length may be determined by aligning the sequences and identifying the conserved regions described herein. 1×SSC is 0.15M NaCl and 15 mM sodium citrate; the hybridization solution and wash solutions may additionally include 5×Denhardt's reagent, 0.5-1.0% SDS, 100 μg/ml denatured, fragmented salmon sperm DNA, 0.5% sodium pyrophosphate. Another example of high stringency conditions is hybridization at 65° C. in 0.1×SSC comprising 0.1 SDS and optionally 5×Denhardt's reagent, 100 μg/ml denatured, fragmented salmon sperm DNA, 0.5% sodium pyrophosphate, followed by the washing at 65° C. in 0.3×SSC.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

As used herein, a “primer” refers to a nucleic acid molecule that can anneal to a template nucleic acid and serves as a starting point for DNA amplification. The primer can be entirely or partially complementary to a specific region of the template polynucleotide, for example 20 nucleotides upstream or downstream from a codon of interest. A non-complementary nucleotide is defined herein as a mismatch. A mismatch may be located within the primer or at the either end of the primer. Preferably, a single nucleotide mismatch, more preferably two, and more preferably, three or more consecutive or not consecutive nucleotide mismatches is (are) located within the primer. The primer can have, for example, from 5 to 200 nucleotides, preferably, from 20 to 80 nucleotides, and more preferably, from 43 to 65 nucleotides. More preferably, the primer has 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, or 190 nucleotides. A “forward primer” as defined herein is a primer that is complementary to a minus strand of the template polynucleotide. A “reverse primer” as defined herein is a primer complementary to a plus strand of the template polynucleotide. Preferably, the forward and reverse primers do not comprise overlapping nucleotide sequences. “Do not comprise overlapping nucleotide sequences” as defined herein means that a forward and reverse primer does not anneal to a region of the minus and plus strands, respectively, of the template polynucleotide in which the plus and minus strands are complimentary to one another. With regard to the primers annealing to the same strand of the template polynucleotide, “do not comprise overlapping nucleotide sequences” means the primers do not comprise sequences complementary to the same region of the same strand of the template polynucleotide. As used herein, a “primer set” refers to a combination of a “forward primer” and a corresponding “reverse primer.”

As used herein, the plus strand equivalent to the sense strand and may also be referred to as a coding or non-template strand. This is the strand that has the same sequence as the mRNA (except it has Ts instead of Us). The other strand, called the template, minus, or antisense strand, is complementary to the mRNA.

As described herein, “codon optimization” refers to the design process of altering codons to codons known to increase maximum protein expression efficiency. In some alternatives, codon optimization for expression in a cell is described, wherein codon optimization can be performed by using algorithms that are known to those skilled in the art so as to create synthetic genetic transcripts optimized for high mRNA and protein yield in a host cell of interest, for example bacterial, fungal, insect, or mammalian cells (including human cells). Codons can be optimized for protein expression in a bacterial cell, mammalian cell, yeast cell, insect cell, or plant cell, for example. Programs containing algorithms for codon optimization in human cells are readily available. Such programs can include, for example, OptimumGene™ or GeneGPS® algorithms. Additionally, codon optimized sequences can be obtained commercially, for example, from Integrated DNA Technologies. In some embodiments, the genes are codon optimized for expression in bacterial, yeast, fungal or insect cells.

“Digestion” of DNA refers to catalytic cleavage of the DNA with a restriction enzyme that acts only at certain sequences in the DNA. The various restriction enzymes used herein are commercially available and their reaction conditions, cofactors and other requirements were used as would be known to the ordinarily skilled artisan. For analytical purposes, typically 1 μg of plasmid or DNA fragment is used with about 2 units of enzyme in about 20 μl of buffer solution. For the purpose of isolating DNA fragments for plasmid construction, typically 5 to 50 μg of DNA are digested with 20 to 250 units of enzyme in a larger volume. Appropriate buffers and substrate amounts for particular restriction enzymes are specified by the manufacturer. Incubation times of about 1 hour at 37° C. are ordinarily used, but may vary in accordance with the supplier's instructions. After digestion the reaction may be electrophoresed on a gel.

The term “heterologous” (or exogenous or foreign or recombinant) polypeptide is defined herein as: a polypeptide that is not native to the host cell. The protein sequence of such a heterologous polypeptide is a synthetic, non-naturally occurring, “man made” protein sequence; a polypeptide native to the host cell in which structural modifications, e.g., deletions, substitutions, and/or insertions, have been made to alter the native polypeptide; or a polypeptide native to the host cell whose expression is quantitatively altered or whose expression is directed from a genomic location different from the native host cell as a result of manipulation of the DNA of the host cell by recombinant DNA techniques, e.g., a stronger promoter.

“Modifications” are described by providing the original amino acid followed by the number of the position within the amino acid sequence. For example, a substitution of amino acid residue 24, means that the amino acid of the parent at position 24, can be substituted with any of the 19 other amino acid residues. In addition, a substitution can be described by providing the original amino acid followed by the number of the position within the amino acid sequence and followed by the specific substituted amino acid. For example, the substitution of histidine at position 120 with alanine is designated as “His120Ala” or “H120A”. Combinations of substitutions are described by inserting comas between the amino acid residues, for example: K24E, D25P, L27H, A141R, G203I, S220L, S398P; represent a combination of seven different amino acid residues substitutions when compared to a parent polypeptide. Variants having substitutions in the context of amino acid changes, may also be applied to nucleic acid modifications, e.g. by substitutions.

As used herein, “transgenic”, “transgene” or “recombinant” means with regard to, for example, a nucleic acid sequence, an expression cassette, genetic construct or a vector comprising the nucleic acid sequence or an organism transformed with the nucleic acid sequences, expression cassettes or vectors, all those constructions brought about synthetically by recombinant or genetechnological methods in which either (a) the nucleic acid sequences comprising desired genetic information to be expressed, or (b) genetic control sequence(s) which is operably linked with the nucleic acid sequence comprising said desired genetic information, for example a promoter, or (c) both (a) and (b), are not located in their natural genetic environment or have been modified by recombinant methods. The natural genetic environment is understood as meaning the natural genomic or chromosomal locus in the original organism. A naturally occurring expression cassette—for example the naturally occurring combination of the natural promoter of the nucleic acid sequences with the corresponding nucleic acid sequence encoding a polypeptide, becomes a transgenic expression cassette when this expression cassette is modified through human intervention such as, for example, mutagenic treatment. Suitable methods are described, for example, in U.S. Pat. No. 5,565,350, US200405323 and WO 00/15815. Furthermore, a naturally occurring expression cassette becomes a recombinant expression cassette when this expression cassette is isolated from its natural genetic environment and subsequently reintroduced in a genetic environment that is not the natural genetic environment.

A “synthetic” or “artificial” compound is produced by in vitro chemical or enzymatic synthesis. It includes, but is not limited to, variant nucleic acids made with optimal codon usage for host organisms, such as a yeast cell host or other expression hosts of choice or variant protein sequences with amino acid modifications, such as e.g. substitutions, compared to the parent protein sequence, e.g. to optimize properties of the polypeptide.

The term “restriction site” refers to a recognition sequence that is necessary for the manifestation of the action of a restriction enzyme, and includes a site of catalytic cleavage. It is appreciated that a site of cleavage may or may not be contained within a portion of a restriction site that comprises a low ambiguity sequence (i.e. a sequence containing the principal determinant of the frequency of occurrence of the restriction site). Thus, in many cases, relevant restriction sites contain only a low ambiguity sequence with an internal cleavage site (e.g. G/AATTC in the EcoRI site) or an immediately adjacent cleavage site (e.g. /CCWGG in the EcoRII site). In other cases, relevant restriction enzymes (e.g. the Eco57I site or CTGAAG(16/14)) contain a low ambiguity sequence (e.g. the CTGAAG sequence in the Eco57I site) with an external cleavage site (e.g. in the N16 portion of the Eco57I site). When an enzyme (e.g. a restriction enzyme) is said to “cleave” a polynucleotide, it is understood to mean that the restriction enzyme catalyzes or facilitates a cleavage of a polynucleotide.

An “ambiguous base requirement” in a restriction site refers to a nucleotide base requirement that is not specified to the fullest extent, i.e. that is not a specific base (such as, in a non-limiting exemplification, a specific base selected from A, C, G and T), but rather may be any one of at least two or more bases. Commonly accepted abbreviations that are used in the art as well as herein to represent ambiguity in bases include the following: R=G or A; Y=C or T; M=A or C; K=G or T; S=G or C; W=A or T; H=A or C or T; B=G or T or C; V=G or C or A; D=G or A or T; N=A or C or G or T.

A “reference sequence” is a defined sequence used as a basis for a sequence comparison; a reference sequence may be a subset of a larger sequence, for example, as a segment of a full-length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window,” as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith (Smith and Waterman, Adv Appl Math, 1981; Smith and Waterman, J Teor Biol, 1981; Smith and Waterman, J Mol Biol, 1981; Smith et al, J Mol Evol, 1981), by the homology alignment algorithm of Needleman (Needleman and Wuncsch, 1970), by the search of similarity method of Pearson (Pearson and Lipman, 1988), by computerized implementations of these algorithms BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The terms “fragment”, “derivative” and “analog” when referring to a reference polypeptide comprise a polypeptide which retains at least one biological function or activity that is at least essentially same as that of the reference polypeptide.

The term “functional fragment” refers to any nucleic acid or amino acid sequence which comprises merely a part of the full length nucleic acid or full length amino acid sequence, respectively, but still has the same or similar activity and/or function. In one embodiment, the fragment comprises at least 70%, at least 80%, at least 85%, at least 90%, at least 95%, at least 96%, at least 97%, at least 98%, at least 99% of the original sequence. In one embodiment, the functional fragment comprises contiguous nucleic acids or amino acids compared to the original nucleic acid or original amino acid sequence, respectively.

The term “pro-form”, “pro-protein”, or “pro-peptide”, refers to a protein precursor, which is an inactive or low activity protein (or peptide) that can be turned into an active or more active form by post-translational modification, such by cleavage or by addition of another peptide or molecule, to produce a mature protein (e.g., to form an enzyme from a pro-enzyme).

The term “gene” means the segment of DNA involved in producing a polypeptide chain; it includes regions preceding and following the coding region (leader and trailer) as well as optionally intervening sequences (introns) between individual coding segments (exons).

As used herein, the term “isolated” means that the material is removed from its original environment (e.g., the natural environment if it is naturally occurring). For example, a naturally-occurring polynucleotide or enzyme present in a living animal is not isolated, but the same polynucleotide or enzyme, separated from some or all of the coexisting materials in the natural system, is isolated. Such polynucleotides could be part of a vector and/or such polynucleotides or enzymes could be part of a composition, and still be isolated in that such vector or composition is not part of its natural environment. As further example, an isolated nucleic acid, e.g., a DNA or RNA molecule, is one that is not immediately contiguous with the 5′ and 3′ flanking sequences with which it normally is immediately contiguous when present in the naturally occurring genome of the organism from which it is derived. Such polynucleotides could be part of a vector, incorporated into a genome of a cell with an unrelated genetic background (or into the genome of a cell with an essentially similar genetic background, but at a site different from that at which it naturally occurs), or produced by PCR amplification or restriction enzyme digestion, or an RNA molecule produced by in vitro transcription, and/or such polynucleotides, polypeptides, or enzymes could be part of a composition, and still be isolated in that such vector or composition is not part of its natural environment.

“Vector” means any kind of construct suitable to carry foreign polynucleotide sequences for transfer to another cell, or for stable or transient expression within a given cell. The term “vector” encompasses any kind of cloning vehicles, such as but not limited to plasmids, phagemids, viral vectors (e.g., phages), bacteriophage, baculoviruses, cosmids, fosmids, artificial chromosomes, or and any other vectors specific for specific hosts of interest. Low copy number or high copy number vectors are also included. Foreign polynucleotide sequences usually comprise a coding sequence, which may be referred to as a “gene of interest.” The gene of interest may comprise introns and exons, depending on the kind of origin or destination of host cell.

As used herein, the term “purified” does not require absolute purity; rather, it is intended as a relative definition. Individual nucleic acids obtained from a library have been conventionally purified to electrophoretic homogeneity. For example, the purified nucleic acids of the present disclosure can be purified from the remainder of the genomic DNA in the organism by at least 10⁴-10⁶ fold. However, the term “purified” also includes nucleic acids which have been purified from the remainder of the genomic DNA or from other sequences in a library or other environment by at least one order of magnitude, typically two or three orders, and more typically four or five orders of magnitude “Purified” means that the material is in a relatively pure state, e.g., at least about 90% pure, at least about 95% pure, or at least about 98% or 99% pure. Preferably “purified” means that the material is in a 100% pure state.

The term “operably linked” means that the described components are in a relationship permitting them to function in their intended manner. For example, a regulatory sequence operably linked to a coding sequence is ligated in such a way that expression of the coding sequence is achieved under condition compatible with the control sequences. As used herein, a promoter sequence is “operably linked to” a coding sequence when RNA polymerase which initiates transcription at the promoter can transcribe the coding sequence into mRNA.

The term “mutations” is defined as alterations in the genetic code of nucleic acid sequence or alterations in the sequence of a peptide. Such mutations may be point mutations such as transitions or transversions. A mutation may be a change to one or more nucleotides or encoded amino acid sequences. The mutations may be deletions, insertions or duplications.

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

For nucleotide sequences, e.g., consensus sequences, an IUPAC nucleotide nomenclature (Nomenclature Committee of the International Union of Biochemistry (NC-IUB) (1984). “Nomenclature for Incompletely Specified Bases in Nucleic Acid Sequences”.) is used, with the following nucleotide and nucleotide ambiguity definitions, relevant to this description: A, adenine; C, cytosine; G, guanine; T, thymine; K, guanine or thymine; R, adenine or guanine; W, adenine or thymine; M, adenine or cytosine; Y, cytosine or thymine; D, not a cytosine; N, any nucleotide.

In addition, notation “N(3-5)” means that indicated consensus position may have 3 to 5 any (N) nucleotides. For example, a consensus sequence “AWN(4-6)” represents 3 possible variants—with 4, 5, or 6 any nucleotides at the end: AWNNNN, AWNNNNN, AWNNNNNN.

The terms “nucleic acid sequence coding for” or a “DNA coding sequence of” or a “nucleotide sequence encoding” a particular protein or polypeptide refer to a DNA sequence which is transcribed and translated into a protein or polypeptide when placed under the control of appropriate regulatory sequences.

The terms “nucleic acid encoding a protein or peptide” or “DNA encoding a protein or peptide” or “polynucleotide encoding a protein or peptide” and other synonymous terms encompasses a polynucleotide which includes only coding sequence for the protein or peptide as well as a polynucleotide which includes additional coding and/or non-coding sequence.

The terms “regulatory element”, “control sequence” and “promoter” are all used interchangeably herein and are to be taken in a broad context to refer to regulatory nucleic acid sequences capable of effecting expression of the sequences to which they are associated. “Regulatory elements” or “regulatory nucleotide sequences” herein may mean pieces of nucleic acid which drive expression of a nucleic acid sequence. upon transformation into a host cell or cell organelle had occurred. Regulatory nucleotide sequences may include any nucleotide sequence having a function or purpose individually and within a particular arrangement or grouping of other elements or sequences within the arrangement. Examples of regulatory nucleotide sequences include but are not limited to transcription control elements such as promoters, enhancers, and termination elements. Regulatory nucleotide sequences may be native (i.e. from the same gene) or foreign (i.e. from a different gene) to a nucleotide sequence to be expressed.

The term “promoter” typically refers to a nucleic acid control sequence located upstream from the transcriptional start of a gene and is involved in recognizing and binding of RNA polymerase and other proteins, thereby directing transcription of an operably linked nucleic acid. “Promoter” herein may further include any nucleic acid sequence capable of driving transcription of a coding sequence. In particular, the term “promoter” as used herein may refer to a polynucleotide sequence generally described as the 5′ regulator region of a gene, located proximal to the start codon. The transcription of one or more coding sequence is initiated at the promoter region. The term promoter may also include fragments of a promoter that are functional in initiating transcription of the gene. Promoter may also be called “transcription start site” (TSS).

Encompassed by the aforementioned terms are further transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner.

For example, enhancers as known in the art and as used herein are normally short DNA segments (e.g. 50-1500 bp) which may be bound by proteins such as transcription factors to increase the likelihood that transcription of a coding sequence will occur.

Also included within the term is a transcriptional regulatory sequence of a classical prokaryotic gene, in which case it may include a -35 box sequence and/or -10 box transcriptional regulatory sequences. The term “regulatory element” also encompasses a synthetic fusion molecule or derivative that confers, activates or enhances expression of a nucleic acid molecule in a cell, tissue or organ. A promoter can be modified by one or more nucleotide substitution(s), insertion(s) and/or deletion(s) without interfering with functionality or activity, but it is also possible to increase the activity by modification of its sequence.

Further elements may be “transcription termination elements” which include pieces of nucleic acid sequences marking the end of a gene and mediating the transcriptional termination by providing signals within mRNA that initiates the release of the mRNA from the transcriptional complex. Transcriptional termination in prokaryotes usually is initiated by Rho-dependent or Rho-independent terminators. In eukaryotes transcription termination usually occurs through recognition of termination by proteins associated with RNA polymerase II.

An “oligonucleotide” (or synonymously an “oligo”) refers to either a single stranded polydeoxynucleotide or two complementary polydeoxynucleotide strands which may be chemically synthesized. Such synthetic oligonucleotides may or may not have a 5′ phosphate. Those that do not will not ligate to another oligonucleotide without adding a phosphate with an ATP in the presence of a kinase. A synthetic oligonucleotide will ligate to a fragment that has not been dephosphorylated.

Any source of nucleic acid, in purified form can be utilized as the starting nucleic acid (also defined as “a template polynucleotide”). Thus, the process may employ DNA or RNA including messenger RNA, which DNA or RNA can be single-stranded, and preferably double stranded. In addition, a DNA-RNA hybrid which contains one strand of each may be utilized. The nucleic acid sequence may be of various lengths depending on the size of the nucleic acid sequence to be mutated. Preferably the specific nucleic acid sequence is from 50 to 50000 base pairs, and more preferably from 50-11000 base pairs. Standard convention (5′ to 3′) is used herein to describe the sequence of double-stranded polynucleotides.

All methods and materials similar or equivalent to those described herein can be used in the practice or testing of methods and compositions disclosed herein, with suitable methods and materials being described herein. All publications, patent applications, patents, and other references mentioned herein are incorporated by reference in their entirety. Further, the materials, methods, and examples are illustrative only and are not intended to be limiting, unless otherwise specified.

Variant polypeptides having an amino acid substitution, wherein the substitution may be a conservative amino acid substitution. A “conservative amino acid substitution” or related amino acid” means replacement of one amino acid residue in an amino acid sequence with a different amino acid residue having a similar property at the same position compared to the parent amino acid sequence. Some examples of a conservative amino acid substitution include but are not limited to replacing a positively charged amino acid residue with a different positively charged amino acid residue; replacing a polar amino acid residue with a different polar amino acid residue; replacing a non-polar amino acid residue with a different non-polar amino acid residue, replacing a basic amino acid residue with a different basic amino acid residue, or replacing an aromatic amino acid residue with a different aromatic amino acid residue. A list of related amino acids is given in the Table below (see for example Creighton (1984) Proteins. W.H. Freeman and Company (Eds)).

The variant polypeptides having G4-amylase activity may be a “mature polypeptide.” A mature polypeptide means an enzyme in its final form including any post-translational modifications, glycosylation, phosphorylation, truncation, N-terminal modifications, C-terminal modifications, signal sequence deletion. A mature polypeptide can vary depending upon the expression system, vector, promoter, and/or production process.

“Enzymatic activity” means at least one catalytic effect exerted by an enzyme. Enzymatic activity is expressed as units per milligram of enzyme (specific activity) or molecules of substrate transformed per minute per molecule of enzyme (molecular activity). Enzymatic activity can be specified by the enzymes actual function, e.g. proteases exerting proteolytic activity by catalyzing hydrolytic cleavage of peptide bonds, lipases exerting lipolytic activity by hydrolytic cleavage of ester bonds, etc.

Enzymatic activity changes during storage or operational use of the enzyme. The term “enzyme stability” relates to the retention of enzymatic activity as a function of time during storage or operation.

To determine and quantify changes in catalytic activity of enzymes stored or used under certain conditions over time, the “initial enzymatic activity” is measured under defined conditions at time cero (100%) and at a certain point in time later (x %). By comparison of the values measured, a potential loss of enzymatic activity can be determined in its extent. The extent of enzymatic activity loss determines an enzymes stability or non-stability.

Parameters influencing the enzymatic activity of an enzyme and/or storage stability and/or operational stability are for example pH, temperature, and presence of oxidative substances. The description also includes nucleic acids and polypeptides optimized for expression in these organisms and species.

“Thermal stability” and “thermostability” refer to the ability of a protein to function over a temperature range. In general, most enzymes have a finite range of temperatures at which they function. In addition to enzymes that work in mid-range temperatures (e.g., room temperature), there are enzymes that are capable of working in very high or very low temperatures. Thermostability is characterized by what is known as the T₅₀ value (also called half-life). The T₅₀ indicates the temperature at which 50% residual activity is still present after thermal inactivation for a certain time compared with a reference sample which has not undergone thermal treatment.

“Thermal tolerance” and “thermotolerance” refer to the ability of a protein to function after exposure to a specific temperature, such as a very high or very low temperature. A thermotolerant protein may not function at the exposure temperature, but will function once returned to a favorable temperature. A substantial change in thermal stability is evidenced by at least about 5% or greater modification (increase or decrease) in the half-life of the enzymatic activity when exposed to given temperature.

“pH stability”, refers to the ability of a protein to function over a specific pH range. In general, most enzymes are working under conditions with rather high or rather low pH ranges.

G4 Amylase polypeptides

In some embodiments amylase polypeptides are provided. Different classes of amylases are known to be useful for industrial application including: Alpha-amylase (E.C. 3.2.1.1); Beta-amylase (E.C. 3.2.1.2); G4-amylase (3.2.1.60), Glucan 1, 4-alpha-maltotetraohydrolase (E.C. 3.2.1.60), also known as exo-maltotetraohydrolase, G4-amylase; Glucan 1,4-alpha-maltohydrolase (E.C. 3.2.1.133), also known as maltogenic alpha-amylase; Endo-1,4-beta-xylanase (E.C. 3.2.1.8); Oxidoreductases; Phospholipase A1 (E.C. 3.1.1.32) Phospholipase A2 (E.C. 3.1.1.4); Phospholipase C (E.C. 3.1.4.3); Phospholipase D (E.C. 3.1.4.4); Galactolipase (E.C. 3.1.1.26), Cellulase (EC 3.2.1.4), Transglutaminases (EC 2.3.2.13), Phytase (EC 3.1.3.8; 3.1.3.26; and 3.1.1.72) and Protease. Enzymes are used as food ingredients, food additives, and/processing aids. Amylase enzymes are disclosed in patents and published patent applications including: WO2002/068589, WO2002/068597, WO2003/083054, WO2004/042006, WO2008/080093, WO2013/116175, and WO2017/106633. Commercial amylase enzymes used in food processing and baking including: Veron® from AB Enzymes; BakeDream®, BakeZyme®, and Panamore® available from DSM; POWERSoft®, Max-LIFE™, POWERFlex®, and POWERFresh® available from DuPont; and Fungamyl®, Novamyl®, OptiCake®, and Sensea® available from Novozymes.

In some embodiment a G4 amylase is provided. G4-amylase (Glucan 1, 4-alpha-maltodextraohydrolase) also known as exo-maltotetraohydrolase Glucan 1,4-alpha-maltotetrahydrolase) are known as maltotetraose-forming amylase, and are used for breaking maltotetraose from sucrose chains. G4 amylases are useful in the generation of syrups containing a high degree of maltotetraose as well as in the generation of modified starch whose amylopectin branches have been specifically trimmed and not completely digested by the G4 amylase. This partially hydrolyzed starch is useful as a texture generating component in foods.

In some embodiment variants of G4 amylase polypeptide are provided wherein the polypeptide has G4 amylase activity. The variant polypeptides may be active over a broad pH at any single point within the range from about pH 4.0 to about pH 12.0. The variant polypeptides having G4-amylase activity are active over a range of pH 4.0 to pH 11.0, pH 4.0 to pH 10.0, pH 4.0 to pH 9.0, pH 4.0 to pH 8.0, pH 4.0 to pH 7.0, pH 4.0 to pH 6.0, or pH 4.0 to pH 5.0. The variant polypeptides having G4-amylase enzyme activity is active at pH 4.0, pH 4.1, pH 4.2, pH 4.3, pH4.4, pH 4.5, pH 4.6, pH 4.7, pH 4.8, pH 4.9, pH 5.0, pH 5.1, pH 5.2, pH 5.3, pH 5.4, pH 5.5, pH 5.6, pH 5.7, pH 5.8, pH 5.9, pH 6.0, pH 6.1, pH 6.2, pH 6.3, pH 6.4, pH 6.5, pH 6.6, pH 6.7, pH 6.8, pH 6.9, pH 7.0, pH 7.1, pH 7.2, pH 7.3, pH 7.4, pH 7.5, pH 7.6, pH 7.7, pH 7.8, pH 7.9, pH 8.0, pH 8.1, pH 8.2, pH 8.3, pH 8.4, pH 8.5, pH 8.6 pH 8.7, pH 8.8 pH 8.9, pH 9.0, pH 9.1, pH 9.2, pH 9.3, pH 9.4, pH 9.5, pH 9.6, pH 9.7, pH 9.8, pH 9.9, pH 10.0, pH 10.1, pH 10.2, pH 10.3, pH 10.4, pH 10.5, pH 10.6, pH 10.7, pH 10.8, pH 10.9, pH 11.0, pH 11.1, pH 11.2, pH 11.3, pH 11.4, pH 11.5, pH 11.6, pH 11.7, pH 11.8, pH 11.9, pH 12.0, pH 12.1, pH 12.2, pH 12.3, pH 12.4, and pH 12.5, pH 12.6, pH 12.7, pH 12.8, pH 12.9, and higher.

Variant polypeptides may be active over a broad temperature used in at any time during a baking process, wherein the temperature is any point in the range from about 20° C. to about 80° C. The variant polypeptides having G4-amylase enzyme activity are active at a temperature range from 20° C. to 55° C., 20° C. to 50° C., 20° C. to 45° C., 20° C. to 40° C., 20° C. to 35° C., 20θ C to 30° C., or 20° C. to 25° C. The variant polypeptides having G4-amylase enzyme activity are active at a temperature of at least 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C. or higher temperatures.

The variant polypeptides are described as an amino acid sequence which is at least n % identical to the amino acid sequence of the respective parent enzyme with “n” being a number between 10 and 100. The variant polypeptides include enzymes that are least 80% identical, at least 80.5%, at least 81%, at least 81.5%, at least 82%, at least 82.5%, at least 83%, at least 83.5%, at least 84%, at least 84.5%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or at least 100% identical when compared to the full length amino acid sequence of the parent enzyme, wherein the enzyme variant has enzymatic activity. In some embodiments the parent enzyme is polypeptide with sequence 1.

In some embodiment a variant of the polypeptide of SEQ ID NO: 1 comprising a modification at one or more, and not exceeding 36 positions and having G4 amylase activity is provided.

The variant polypeptides having G4-amylase activity may be a hybrid of more than one G4-amylase enzyme. A “hybrid” or “chimeric” or “fusion protein” means that a domain of a first variant polypeptides G4-amylase is combined with a domain of a second G4-amylase to form a hybrid amylase and the hybrid has amylase activity. A domain of variant polypeptides having G4 amylase enzyme activity can be combined with a domain of a commercially available amylase, such as Veron® from AB Enzymes; BakeDream®, BakeZyme®, and Panamore® available from DSM; POWERSoft®, Max-LIFE™, POWERFlex®, and POWERFresh® available from DuPont; and Fungamyl®, Novamyl®, OptiCake®, and Sensea® available from Novozymes. In addition, domains from various amylase enzymes can be recombined into a single enzyme, wherein the enzyme has amylase activity.

In some embodiment, the G4 amylase is a recombinant protein produced using bacteria, fungi, or yeast expression systems. “Expression system” here means a host microorganism, expression hosts, host cell, production organism, or production strain and each of these terms can be used interchangeably. Examples of expression systems include but are not limited to: Aspergillus niger, Aspergillus oryzae, Hansenula polymorpha, Thermomyces lanuginosus, Fusarium oxysporum, Fusarium heterosporum, Escherichia coli, Bacillus, preferably Bacillus subtilis, or Bacillus licheniformis, Pseudomonas, preferably Pseudomonas fluorescens, Pichia pastoris (also known as Komagataella phaffii), Myceliopthora thermophile (C1), Schizosaccharomyces pombe, Trichoderma, preferably Trichoderma reesei and Saccharomyces, preferably Saccharomyces cerevisiae. The variant polypeptides having G4-amylase enzyme activity are produced using the expression system listed above. In one embodiment, a method of making the variant polypeptides having G4-amylase enzyme activity comprises: providing a template nucleic acid sequence wherein the template nucleic acid is a variant polypetide of the sequence as set forth in SEQ ID NO:1, wherein the variant polypeptide has G4-amylase activity.

In one embodiment, the variant polypeptide has at least 5% higher residual activity at residual activity at 65° C. in comparison with Seq 1.

The polypeptide variants having G4-amylase enzyme activity may be used or formulated alone or as a mixture of enzymes. The formulation may be a solid form such as powder, a lyophilized preparation, a granule, a tablet, a bar, a crystal, a capsule, a pill, a pellet, or in a liquid form such as in an aqueous solution, an aerosol, a gel, a paste, a slurry, an aqueous/oil emulsion, a cream, a capsule, or in a vesicular or micellar suspension.

The variant polypeptides having G4-amylase enzyme activity may be used in combination with at least one other enzyme. The other enzyme may be from the same class of enzymes, for example, a composition comprising a first G4-amylase and a second G4-amylase. The other enzyme may also be from a different class of enzymes, for example, a composition comprising the variant polypeptides having G4-amylase enzyme activity and a lipase. The combination with at least one other enzyme may be a composition comprising at least three enzymes. The three enzymes may have enzymes from the same class of enzymes, for example a first amylase, a second amylase, and a third amylase; or the enzymes may be from different class of enzymes for example, the variant polypeptides having G4-amylase enzyme activity, a lipase, and a xylanase.

The second enzyme comprises: an alpha-amylase; a G4-amylase, a Glucan 1, 4-alpha-maltotetraohydrolase, also known as exo-maltotetraohydrolase, beta-amylase; a Glucan 1,4-alpha-maltohydrolase, also known as maltogenic alpha-amylase, a cyclodextrin glucanotransferase, a glucoamylase; an Endo-1,4-beta-xylanase; a xylanase, a cellulase, an Oxidoreductases; a Phospholipase A1; a Phospholipase A2; a Phospholipase C; a Phospholipase D; a Galactolipase, triacylglycerol lipase, an arabinofuranosidase, a transglutaminase, a pectinase, a pectate lyase, a protease, or any combination thereof. The enzyme combination is the variant polypeptides having G4-amylase enzyme activity and a lipase, or the enzyme combination is the G4-amylase, a lipase, and a xylanase. Amylases in combination with other enzymes, like lipases can be used for industrial application. Lipases (E.C. 3.1.1.3) are hydrolytic enzymes that are known to cleave ester bonds in lipids. Lipases include phospholipases, triacylglycerol lipases, and galactolipases.

Lipases have been identified from plants; mammals; and microorganisms including but not limited to: Pseudomonas, Vibrio, Acinetobacter, Burkholderia, Chromobacterium, Cutinase from Fusarium solani (FSC), Candida antarctica A (CalA), Rhizopus oryzae (ROL), Thermomyces lanuginosus (TLL), Rhizomucor miehei (RML), Aspergillus Niger, Fusarium heterosporum, Fusarium oxysporum, Fusarium culmorum lipases. In addition, many lipases, phospholipases, and galactolipases have been disclosed in patents and published patent applications including, but not limited to: WO1993/000924, WO2003/035878, WO2003/089620, WO2005/032496, WO2005/086900, WO2006/031699, WO2008/036863, and WO2011/046812. Commercial lipases used in food processing and baking including, but not limited to: LIPOPAN™ NOOPAZYME, LIPOPAN MAX, LIPOPAN Xtra (available from Novozymes); PANAMORE, CAKEZYME, and BAKEZYME (available from DSM); and GRINDAMYL EXEL 16, GRINDAMYL POWERBAKE, and TS-E 861 (available from Dupont/Danisco).

The variant polypeptides having G4-amylase enzyme activity may be in a composition. The composition comprises the variant polypeptides having G4-amylase enzyme activity and a second enzyme. Preferably the second enzyme is selected from the group consisting of: a second G4-amylase, a lipase, an alpha-amylase, a G4-amylase, a xylanase, a protease, a cellulase, a glucoamylase, an oxidoreductase, a phospholipase, and a cyclodextrin glucanotransferase. The composition may be used in the preparation of bakery products.

The variant polypeptides having G4-amylase enzyme activity may be used in a method of preparing a dough or a beaked product prepared from the dough, the method comprising adding one of the variant enzymes to the dough and baking it. Bread includes, but is not limited to: rolls, buns, pastries, cakes, flatbreads, pizza bread, pita bread, wafers, pie crusts naan, lavish, pitta, focaccia, sourdoughs, noodles, cookies, deep-fired (doughnuts) tortillas, pancakes, crepes, croutons, and biscuits. The bread could also be an edible container such as a cup or a cone. Baking bread generally involves mixing ingredients to form dough, kneading, rising, shaping, baking, cooling and storage. The ingredients used for making dough generally include flour, water, salt, yeast, and other food additives.

Flour is generally made from wheat and may be milled for different purposes such as making bread, pastries, cakes, biscuits pasta, and noodles. Alternatives to wheat flour include, but are not limited to: almond flour, coconut flour, chia flour, corn flour, barley flour, spelt flour, soya flour, hemp flour, potato flour, quinoa, teff flour, rye flour, amaranth flour, arrowroot flour, chick pea (garbanzo) flour, cashew flour, flax meal, macadamia flour, millet flour, sorghum flour, rice flour, tapioca flour, and any combination thereof. Flour type is known to vary between different regions and different countries around the world.

Treatment of flour or dough, may include adding inorganic substances, organic substances such as fatty acids, carbohydrates, amino acids, proteins, and nuts. The flour or dough may be pretreated prior to baking by cooling, heating, irradiation, agglomeration, or freeze-drying. In addition, the flour or dough may be pretreated prior to baking by adding enzymes, or micro-organisms, such as yeasts.

Yeast breaks down sugars into carbon dioxide and water. A variety of Baker's yeast, which are usually derived from Saccharomyces cerevisiae, are known to those skilled in the art including, but not limited to: cream yeast, compressed yeast, cake yeast, active dry yeast, instant yeast, osmotolerant yeasts, rapid-rise yeast, deactivated yeast. Other kinds of yeast include nutritional yeast, brewer's yeast, distiller's and wine yeast.

Sweeteners include but are not limited to: liquid sugar, syrups, white (granulated) sugars, brown (raw) sugars, honey, fructose, dextrose, glucose, high fructose corn syrup, molasses, and artificial sweeteners.

Emulsifiers include but are not limited to diacetyl tartaric acid esters of monoglycerides (DATEM), sodium stearoyl lactylate (SSL), calcium stearoyl lactylate (CSL), ethoxylated mono- and diglycerides (EMG), polysorbates (PS), distilled monoglycerides (DMG) and succinylated monoglycerides (SMG).

Other food additives may be used with the methods of baking include: Lipids, oils, butter, margarine, shortening, butterfat, glycerol, eggs, diary, non-diary alternatives, thickeners, preservatives, colorants, and enzymes.

Ingredients or additives for baking may be added individually to during the baking process. The ingredients or additives may also be combined with more than one ingredient or additive to form pre-mixes and then the pre-mixes are added during the baking process. The flour or dough mixtures may be prepared prior to baking including ready-for oven doughs, packaged doughs or packaged batters.

Bakery products may be modified to meet special dietary requirements such as sugar-free, gluten-free, low fat, or any combination thereof. The enzymes may extend shelf-life of a dough-based product or provide antimicrobial (mold-free) effects.

“Baked products” includes baked products such as bread, crispy rolls, sandwich bread, buns, baguette, ciabatta, croissants, noodles, as well as fine bakery wares like donuts, brioche, stollen, cakes, muffins, etc. “Dough” is defined as a mixture of flour, salt, yeast and water, which may be kneaded, molded, shaped or rolled prior to baking. In addition, also other ingredients such as sugar, margarine, egg, milk, etc. might be used. The term includes doughs used for the preparation of baked goods, such as bread, rolls, sandwich bread, baguette, ciabatta, croissants, sweet yeast doughs, etc. “Bread volume” is the volume of a baked good determined by using a laser scanner (e.g. Volscan Profiler ex Micro Stable System) to measure the volume as well as the specific volume. The term also includes the volume which is determined by measuring the length, the width and the height of certain baked goods.

The description in range format is merely for convenience and brevity and should not be construed as an inflexible limitation on the scope of the disclosure. Accordingly, the description of a range is considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Variant polypeptides having G4-amylase enzyme activity may be useful for other industrial applications. The variant polypeptides having G4-amylase enzyme activity are used in a detergent, a personal care product, in the processing of textiles, in pulp and paper processing, in the production of ethanol, lignocellulosic ethanol, or syrups; as viscosity breakers in oilfield and mining industries. The variant polypeptides are active on raw starch as well.

In the following description, numerous specific details are set forth to provide a more thorough understanding of the present disclosure. However, it will be apparent to one of skill in the art that the methods of the present disclosure may be practiced without one or more of these specific details. In other instances, well-known features and procedures well known to those skilled in the art have not been described to avoid obscuring the disclosure.

### Example 1: PAHBAH Assay

Quantitation of starch hydrolysis for the G4-amylase and variant enzymes was measured using the 4-Hydroxybenzhydrazide method as described in Lever M. (1972) “A new reaction for colorimetric determination of carbohydrates.” Anal. Biochem. 47, 273-279, with the following modifications. 112 uL of 1% potato amylopectin was reacted with 12.5 uL of diluted enzyme at 65° C. and samples taken at 10 minutes. The reaction was then quenched by mixing into 100 ul 1% PAHBAH reagent. The reaction was heated to 95° C. for 6 minutes, cooled to room temperature, and the solution absorption was read at 410 nm in a BioTek plate reader.

### Example 2: Residual Activity

Residual activity was calculated by comparing the activity of each enzyme as measured using the PAHBAH assay before and after a heat challenge at 71° C. After heating the sample for 6.5 minutes at 71° C., the sample was cooled to room temperature before being tested, using the PAHBAH assay, at 65° C.

### Example 3: ThermoFluor Assay

The melting point (Tₘ) for each G4-amylase enzyme variant was measured in a high throughput manner using the ThermoFluor assay as described in Lo, MC; Aulabaugh, A; Jin, G; Cowling, R; Bard, J; Malamas, M; Ellestad, G (1 Sep. 2004). “Evaluation of fluorescence-based thermal shift assays for hit identification in drug discovery” Analytical Biochemistry 332 (1): 153-9.:10.1016/j.ab.2004.04.031, PMID 15301960, with the following modifications. A 50 ul reaction in 50 mM Na Acetate pH6, 5×SYPRO orange and enzyme supernatant was subjected to at heat ramp from 50° C. to 98° C. at 1° C. per minute. The dye fluorescence was monitored using in a BioRad CFX 384 real time PCR machine. The melt curve data was analyzed using the supplied CFX Manager Software.

### Example 4: Expression of Variant G4-Amylase

The variant polypeptides having G4-amylase activity were obtained by constructing expression plasmids containing the encoding polynucleotide sequences, transforming plasmids into Pichia pastoris (Komagataella phaffii) and growing the resulting expression strains in the following way. Fresh Pichia pastoris cells of the expression strains were obtained by spreading the glycerol stocks of sequence-confirmed strains onto Yeast extract Peptone Dextrose (YPD) agar plates containing Zeocin. After 2 days, starter seed cultures of the production strains were inoculated into 100 mL of Buffered Glycerol complex Medium (BMGY) using cells from these plates, and grown for 20-24 hours at 30° C. and 225-250 rpm. Seed cultures were scaled up by transferring suitable amounts into 2-4 L of BMMY medium in a baffled Fermenter. Fermentations were carried out at 30° C. and under 1100 rpm of agitation, supplied via flat-blade impellers, for 48-72 hours. After the initial batch-phase of fermentation, sterile-filtered Methanol was added as feed whenever the dissolved oxygen level in the culture dipped below 30%. Alternatively, feed was added every 3 hours at 0.5% v/v of the starting batch culture. The final fermentation broth was centrifuged at 7000×g for 30 mins at 4° C. to obtain the cell-free supernatant.

The variant polypeptides having G4-amylase amylase were identified as follows: supernatant was assayed for protein of interest expression by either SDS-PAGE or capillary electrophoresis.

### Example 5: Variant G4-amylase Enzymes

A parent enzyme was selected identified in this application as the amino acid sequence of SEQ ID NO:1, encoded by the nucleic acid sequence SEQ ID NO.:2. The parent enzyme was engineered in the lab to generate non-naturally occurring G4-amylase variant enzymes having improve characteristics of the enzyme. The improved characteristics include thermostability, enzyme activity, or any combination thereof.

The variant polypeptide enzymes were created starting with the parent enzyme and evolving it using Gene Site Saturation Mutagenesis (GSSM) of the parent enzyme as described in at least U.S. Pat. Nos. 6,562,594, 6,171,820, and 6,764,835; Error Prone PCR; and/or Tailored Multi-Site-Combinatorial Assembly (TMSCA), as described in U.S. Pat. No. 9,476,078.

The G4-amylase variant enzymes may be an amino acid insertion, deletion, or substitution when compared to the parent enzyme. The substitutions may be one amino acid residue for a different amino acid; the substitutions may be conservative amino acid substitutions, or the substitutions may be a similar amino acid residue. In addition, the substitutions may be at more than one site in the amino acid sequence. For example, a G4-amylase variant may have at least two amino acid substitutions wherein the substitutions are at amino acid residues 31 and 190. In addition, multiple substitutions may be made to the parent enzyme. For example, a G4-amylase variant may have amino acid substitutions at amino acid residue numbers 206, 348 and 422. Additional examples the structural changes to the amino acid sequence of the parent enzyme and having single point amino acid substitutions or combinations of amino acid substitutions, and the functional improvements of the variant polypeptides having improved enzyme activity, thermostability, pH, and any combination thereof when compared to the parent enzyme are shown below in Table 1.

TABLE 1

| | | PAHBAH | | | |
|---|---|---|---|---|---|
| | | Residual | | | |
| | PAHBAH | Activity | % | Tm | |
| Variant | at 65° C. | at 65° C. | Residual | (° C.) | Enzymes |
| Seq 1/2 | 2.902 | 0.517 | 17.8 | 55.5 | Parent |
| VAR001 | 0.755 | 0.295 | 39.1 | 58.0 | T31C- |
| | | | | | G190R |
| VAR002 | 1.248 | 0.387 | 31.0 | 57.0 | P111T |
| VAR003 | 1.256 | 0.4 | 31.8 | 58.0 | G123F |
| VAR004 | 1.319 | 0.336 | 25.5 | 57.5 | G136M |
| VAR005 | 1.439 | 0.406 | 28.2 | 59.0 | G136R |
| VAR006 | 0.971 | 0.325 | 33.5 | 59.5 | G136K |
| VAR007 | 1.047 | 0.346 | 33.0 | 62.0 | G136C |
| VAR008 | 0.24 | 0.24 | 100.0 | 61.5 | D141M |
| VAR009 | 0.273 | 0.268 | 98.2 | 61.0 | D141I |
| VAR010 | 0.257 | 0.245 | 95.3 | 60.5 | D141P |
| VAR011 | 1.51 | 0.431 | 28.5 | 60.5 | T143E |
| VAR012 | 1.348 | 0.471 | 34.9 | 68.0 | T143P |
| VAR013 | 1.239 | 0.4 | 32.3 | 58.5 | T143A |
| VAR014 | 1.292 | 0.425 | 32.9 | 58.5 | T143Y |
| VAR015 | 1.476 | 0.388 | 26.3 | 62.0 | T143F |
| VAR016 | 1.535 | 0.459 | 29.9 | 59.0 | P145Y |
| VAR017 | 1.227 | 0.425 | 34.6 | 59.0 | Y148W |
| VAR018 | 0.972 | 0.398 | 40.9 | 59.5 | A149P |
| VAR019 | 0.256 | 0.244 | 95.3 | 71.0 | C152P |
| VAR020 | 0.241 | 0.234 | 97.1 | 61.5 | C152T |
| VAR021 | 0.24 | 0.237 | 98.8 | 61.5 | D153S |
| VAR022 | 0.261 | 0.242 | 92.7 | 68.0 | D156G |
| VAR023 | 0.943 | 0.364 | 38.6 | 57.5 | G160N |
| VAR024 | 1.569 | 0.373 | 23.8 | 55.5 | G160M |
| VAR025 | 1.128 | 0.356 | 31.6 | 58.5 | D162E |
| VAR026 | 0.855 | 0.327 | 38.2 | 55.5 | D178N |
| VAR027 | 1.217 | 0.347 | 28.5 | 55.0 | A181E |
| VAR028 | 0.962 | 0.319 | 33.2 | 56.5 | A181N |
| VAR029 | 1.09 | 0.349 | 32.0 | 55.0 | A181R |
| VAR030 | 0.935 | 0.323 | 34.5 | 56.0 | R184A |
| VAR031 | 0.797 | 0.333 | 41.8 | 56.5 | H213D |
| VAR032 | 0.898 | 0.316 | 35.2 | 56.5 | N215P |
| VAR033 | 0.806 | 0.549 | 68.1 | 57.0 | G227S |
| VAR034 | 0.872 | 0.502 | 57.6 | 56.5 | G227D |
| VAR035 | 0.642 | 0.424 | 66.0 | 56.5 | G227H |
| VAR036 | 1.177 | 0.379 | 32.2 | 56.5 | G227E |
| VAR037 | 1.066 | 0.341 | 32.0 | 56.0 | G227N |
| VAR038 | 0.938 | 0.347 | 37.0 | 57.5 | G227W |
| VAR039 | 0.869 | 0.356 | 41.0 | 55.5 | G227Q |
| VAR040 | 0.756 | 0.31 | 41.0 | 57.0 | H309R |
| VAR041 | 0.899 | 0.382 | 42.5 | 56.5 | H309K |
| VAR042 | 0.741 | 0.459 | 61.9 | 58.5 | A315S |
| VAR043 | 0.764 | 0.467 | 61.1 | 59.0 | A315C |
| VAR044 | 0.739 | 0.457 | 61.8 | 56.0 | A315Y |
| VAR045 | 0.594 | 0.401 | 67.5 | 57.0 | D345E |
| VAR046 | 0.974 | 0.322 | 33.1 | 56.0 | F346L |
| VAR047 | 0.696 | 0.441 | 63.4 | 55.5 | S394E |
| VAR048 | 1.131 | 0.35 | 30.9 | 55.0 | S401Y |
| VAR049 | 0.732 | 0.446 | 60.9 | 56.5 | A206D- |
| | | | | | R348G- |
| | | | | | Q422R |

Although the present disclosure has been described with reference to specific embodiments, these embodiments are illustrative only and not limiting. Many other applications and embodiments of the present disclosure will be apparent in light of this description and the following claims.

1. A variant polypeptide having G4 amylase activity selected from the group consisting of:
(a) a polypeptide having at least 80% sequence identity with SEQ ID NO: 1;
(b) a polypeptide encoded by a polynucleotide having at least 80% sequence identity with SEQ ID NO: 2;
(c) a polypeptide encoded by a polynucleotide that hybridizes under high stringency conditions with (i) a polynucleotide that encodes the amino acid sequence of SEQ ID NO: 1, or (ii) the full-length complement of (i);
(d) a variant of the polypeptide of SEQ ID NO: 1 comprising a modification at one or more, and not exceeding 36 positions and having G4 amylase activity;
(e) a polypeptide encoded by a polynucleotide that differs from SEQ ID NO: 1 due to the degeneracy of the genetic code; and
(f) a fragment of the polypeptide (a), (b), (c), (d) or (e) having G4 amylase activity; wherein the variant amylase has G4 amylase activity.

2. The variant polypeptide of claim 1, wherein the modification is a substitution, deletion, and/or insertion of amino acids.

3. The variant polypeptide of claim 1, wherein the modification is a substitution.

4. The variant polypeptide of claim 1, wherein the modification is at an amino acid residue position number: 31, 111, 123, 136, 141, 143, 145, 148, 149, 152, 153, 156, 160, 162, 178, 181, 184, 190, 206, 213, 215, 227, 309, 315, 345, 346, 348, 394, 401, 422, or any combination thereof.

5. The variant polypeptide of claim 1, wherein the modification is an amino acid substitution selected from T31C, P111T, G123F, G136M, G136R, G136K, G136C, D141M, D141I, D141P, T143E, T143P, T143A, T143Y, T143F, P145Y, Y148W, A149P, C152P, C152T, D153S, D156G, G160N, G160M, D162E, D178N, A181E, A181N, A181R, R184A, G190R, A206D, H213D, N215P, G227S, G227D, G227H, G227E, G227N, G227W, G227Q, H309R, H309K, A315S, A315C, A315Y, D345E, F346L, R348G, S394E, S401Y, Q422R, or any combinations thereof.

6. The variant polypeptide of claim 1, wherein the variant polypeptide is an amino acid sequence that is at least 80% identical, at least 80.5%, at least 81%, at least 81.5%, at least 82%, at least 82.5%, at least 83%, at least 83.5%, at least 84%, at least 84.5%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or at least 100% identical to the amino acid sequence as set forth in SEQ ID NO:1.

7. The variant polypeptide of claim 1, wherein the variant polypeptide is encoded by a polynucleotide having at least 80% identical, at least 80.5%, at least 81%, at least 81.5%, at least 82%, at least 82.5%, at least 83%, at least 83.5%, at least 84%, at least 84.5%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or at least 100% identical to the SEQ ID NO: 2.

8. The variant polypeptide of claim 1, wherein the amino acid substitution is at positions selected from T31C, P111T, G123F, G136M, G136R, G136K, G136C, D141M, D141I, D141P, T143E, T143P, T143A, T143Y, T143F, P145Y, Y148W, A149P, C152P, C152T, D153S, D156G, G160N, G160M, D162E, D178N, A181E, A181N, A181R, R184A, G190R, A206D, H213D, N215P, G227S, G227D, G227H, G227E, G227N, G227W, G227Q, H309R, H309K, A315S, A315C, A315Y, D345E, F346L, R348G, S394E, S401Y, Q422R, or any combinations thereof.

9. The variant polypeptide of claim 1, wherein the variant polypeptide has an increase in enzyme activity, thermostability, or any combination thereof when compared to the G4-amylase of SEQ ID NO:1.

10. A variant polypeptide having G-4-amylase activity, wherein the variant polypeptide is an amino acid sequence that is at least 80% identical, at least 80.5%, at least 81%, at least 81.5%, at least 82%, at least 82.5%, at least 83%, at least 83.5%, at least 84%, at least 84.5%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or at least 100% identical to the amino acid sequence as set forth in SEQ ID NO:1, and the variant polypeptide has an increase in enzyme activity, thermostability, or any combination thereof when compared to the G4-amylase of SEQ ID NO:1.

11. The variant polypeptide comprising a hybrid of at least one variant polypeptide as in claim 1, and a second polypeptide having amylase activity, wherein the hybrid has G4-amylase activity.

12. A composition comprising the variant polypeptide as in claim 1.

13. A composition comprising the variant polypeptide as in claim 1, and a second enzyme.

14. The composition of claim 13, wherein the second enzyme is selected from the group consisting of: a second G4-amylase, a lipase, an alpha-amylase, a beta-amylase, a xylanase, a protease, a cellulase, a glucoamylase, an oxidoreductase, a phospholipase, and a cyclodextrin glucanotransferase.

15. A method of making a variant polypeptide comprising: providing a template nucleic acid sequence encoding the polypeptide variant as in claim 1, transforming the template nucleic acid sequence into an expression host, cultivating the expression host to produce the variant polypeptide, and purifying the variant polypeptide.

16. The method of claim 15, wherein the template nucleic acid is a variant nucleotide of the nucleic acid sequence as set forth in SEQ ID NO:1, wherein the variant nucleotide is a nucleic acid sequence that is at least 80% identical to the nucleic acid sequence as set forth in SEQ ID NO:1, wherein the variant nucleotide encodes a polypeptide having G4-amylase activity.

17. The method of claim 16, wherein the expression host is selected from the group consisting of: a bacterial expression system, a yeast expression system, a fungal expression system, and a synthetic expression system.

18. The method of claim 16, wherein the bacterial expression system is selected from an E. coli, a Bacillus, a Pseudomonas, and a Streptomyces.

19. The method of claim 16, wherein the yeast expression system is selected from a Candida, a Komagataella, a Saccharomyces, a Schizosaccharomyces.

20. The method of claim 16, wherein the fungal expression system is selected from a Penicillium, an Aspergillus, a Fusarium, a Myceliopthora, a Rhizomucor, a Rhizopus, a Thermomyces, and a Trichoderma.

21. A method of preparing a dough or a baked product prepared from the dough, the method comprising adding one of the variant polypeptides as claim 1, to the dough and baking it.

22.-28. (canceled)

29. The variant polypeptide of claim 1, wherein the variant polypeptide has at least 5% higher residual activity at residual activity at 65° C. in comparison with SEQ ID NO:1.


SEQUENCE LISTING (2 sequences)

SEQ ID NO: 1 - protein, 429 aa, Artificial Sequence
  Synthetic Generation
ADAPGKTASGVRYHGGDEIILQGFHWNTVRTSSNWYATLASMAPTLAADGFSAIWMPVPWRDFSSWSDPGNGTSGGGEGYFWHDFNKNGRYGSDSLLRQAAGALNAAGVKPIYDVVPNHMNRGYPDKEINLPAGQGLWRHDCTDPGNYANDCDDGDRFMGGDADLNTGHPQNYAMFRDEFARLRSEYGAGGFRFDFVRGYAGERVASWMSDAHDNGFCLGELWKAPGEYPSWDWRNGASWQQILKDWSDRAKCTVFDFALKERMQNGGIADWRHGLNGNPDARWREVAVTFVDNHDTGYSPGPHGGQHHWPLPDARLKQAYAYILSSPGTPVVYWPHMYDWGHGDFIRQLIQIRRAAGVKAASAIQFHSGFSGLVATISGSQQQLLIALDSNLSSPGQVASGDFTQALNTDNGAIRIWRSGQGGGDEPG

SEQ ID NO: 2 - DNA, 1816 bp, Artificial Sequence
  Synthetic Generation
agatttagaaggggatttcgatgttgctgttttgccattttccaacagcacaaataacgggttattgtttataaatactactattgccagcattgctgctaaagaagaaggggtatctctcgagaaaagagaggctgccgacgcgccgggcaagaccgccagcggcgtgcgctatcacggcggcgacgagatcatcctccagggctttcactggaataccgtgcggacctcgagcaactggtacgcgacgttggccagcatggcgccgaccctggccgccgatggcttcagtgcgatctggatgccggtgccctggcgcgacttctccagctggagcgaccccggcaacggcacctcgggcggtggcgaaggctacttctggcacgacttcaacaagaacggccgctatggcagcgacagcctgctcagacaggccgccggcgcgctcaatgcggccggggtcaaacccatctacgacgtggtgcccaaccacatgaaccgcggctacccggacaaggaaatcaacctgccggccggccaggggctgtggcgccacgactgcactgacccgggcaattacgccaacgactgcgacgacggcgaccgtttcatgggcggcgacgccgacctcaataccggccacccgcagaactacgcgatgttccgcgacgagttcgccaggctgcgcagcgagtatggcgccgggggctttcgcttcgacttcgtccgcggctacgccggcgagcgggtcgccagctggatgagcgatgcccacgacaacggcttctgcctcggcgagctgtggaaggcgccgggcgagtacccgagctgggactggcgcaacggcgccagctggcagcagatcctcaaggactggtccgatcgcgccaagtgcacggtgttcgacttcgccctcaaggagcgcatgcagaacggtggcatcgccgactggcgccacggcctcaacggcaaccccgatgcgcgctggcgcgaggtggcggtgaccttcgtcgacaaccacgacaccggctactcgcccggcccgcacggcggccagcaccactggccgctgccggatgcgcggctgaagcaggcctatgcctacatcctcagcagcccgggtacgccggtggtgtactggccacacatgtacgactggggtcatggcgacttcatccgccagttgatccagatccgccgtgcggccggagtcaaggcggcctcggcgatccagttccactcggggttctccggcctggtggcgaccatcagcggcagccagcagcaactgctgatcgccctggactccaacctgtcctcgcccggccaggtggccagcggcgatttcacccaggcgctcaacaccgacaacggcgcgatccgtatctggcgcagcggtcagggcggcggggatgagccgggcaacctggtcagcgtcaacttccgttgcgacaacggcgtgacccagtggggcgacagcgtctacgcgctgggcaacgtcgcccagctgggcaactggagcccggccggcgccgtgcgcctgaccgacaccagcgcctatccaacctggaagggcagcattgccctgccggccggtcagcaggtgcagtggaagtgcatcgtgcgcagcgaaagcaacccgacccaggtcaagacctggcagccgggcggcaacaacagcgtgacggtggcggcaggagccagtaccgcggggagtttctaatctagaacaaaaactcatctcagaagaggatctgaatagcgccgtcgaccatcatcatcatcatcattgagtttgtagccttagacatgactg